(19) United States
(12) Patent Application Publication
(10) Pub. No.: US 2011/0027339 A1
(43) Pub. Date: Feb. 3, 2011

(54) POROUS IMPLANTS AND STENTS AS CONTROLLED RELEASE DRUG DELIVERY CARRIERS

(76) Inventor: Jeremy J. Mao, Closter, NJ (US)

Correspondence Address: SNR DENTON US LLP, P.O. BOX 061080, CHICAGO, IL, 60606-1080, US

(21) Appl. No.: 12/668,647

(22) PCT Filed: Jul. 10, 2008
(86) PCT No.: PCT/US08/69607

§ 371 Date: Oct. 4, 2010

Related U.S. Application Data
(60) Provisional application No. 60/948,969, filed on Jul. 10, 2007.

(51) Int. Cl. A61F 2/00; A61K 35/12; A61K 38/18; A61K 31/7088; A61P 37/02; A61P 43/00

(52) U.S. Cl. 424/425; 424/424; 424/93.7; 424/278.1; 514/7.6; 514/44 R; 514/8.9

(57) ABSTRACT

The common premise of synthetic implants in the restoration of diseased tissues and organs is to use inert and solid materials. Here, a porous titanium implant enables the delivery of microencapsulated bioactive cues. Control-released TGFβ1 promoted the proliferation and migration of human mesenchymal stem cells into porous implants in vitro. Upon 4-wk implantation in the rabbit humerus, control-released TGFβ1 from porous implants significantly increased BIC by 96% and bone ingrowth by 50% over placebos. Control-released 100 ng TGFβ1 induced equivalent BIC and bone ingrowth to adsorbed 1 μg TGFβ1, suggesting that controlled release is effective at 10-fold less drug dose than adsorption. Histomorphometry, SEM and μT showed that control-released TGFβ1 enhanced bone ingrowth in the implant's pores and surface. These findings suggest that solid prostheses can be transformed into porous implants to serve as drug delivery carriers, from which control-released bioactive cues augment host tissue integration.

### CROSS-REFERENCE TO RELATED APPLICATIONS

This application claims the benefit of U.S. provisional application Ser. No. 60/948,969,filed Jul. 10, 2007, incorporated herein by reference in its entirety.

### STATEMENT REGARDING FEDERALLY SPONSORED RESEARCH OR DEVELOPMENT

This invention was made in part with Government support under National Institute of Biomedical Imaging and Bioengineering and National Institute of Dental and Craniofacial Research Grant Nos. R01DE15391 and R01EB02332. The Government has certain rights in the invention.

### FIELD OF THE INVENTION

The present invention generally relates to drug-delivering implants and stents.

### BACKGROUND

Tissue and organ defects resulting from trauma, chronic diseases, tumor resection or congenital anomalies often necessitate restoration of lost anatomical structures. In contrast with the donor site morbidity and pain associated with autologous tissue grafting, grafts composed of synthetic materials have the advantage of a ready supply without any sacrifice of donor tissue. Previously, synthetic tissue implants have utilized inert and bulk materials to allow the integration of host tissue. Although this has resulted in a number of successful tissue replacement devices including cardiac stents, total joint prosthesis and dental implants, several limitations have become apparent, such as short implant life span and a lack of remodeling with host tissue. Implant failures can be attributed to several causes, though aseptic disintegration is the most common (Sumner, D. R., Turner, T. M. & Urban, R. M. Animal models relevant to cementless joint replacement. J. Musculoskelet. Neuronal. Interact. 1, 333-345 (2001)). Synthetic implants are subject to wear and tear, and do not remodel with host tissue such as cardiac muscle or bone (Misch, C. E. Contemporary Implant Dentistry. (Mosby, Chicago; 1993). Additionally, there is often a mismatch of mechanical properties between synthetic implants and host tissue. For example, titanium (Ti) is approximately 10 times stiffer than cortical bone and 100 times stiffer than cancellous bone ((Millenium Research Group), Toronto, ON, Canada; (2005); Branson, J. J. & Goldstein, W. M. Primary total hip arthroplasty. AORN J. 78, 947-953, 956-969; (2003)). This disparity in mechanical stiffness between Ti and host bone creates stress shielding by diverting functioning mechanical stress, necessary for the health of peri-implant bone, to the Ti implant. Stress shielding leads to osteoclastogenesis and osteolysis (McCarthy, E. F. & Frassica, F. J. Pathology of Bone and Joint Disorders. (W.B. Saunders Company, Philadelphia; 1998); An, Y. H. in Mechanical Testing of Bone and the Bone-implant Interface. (eds. Y. H. An & R. A. Draughn) 41-63 (CRC Press, New York; 2000); Ratner, B. D., Hoffman, A. S., Schoen, F. J. & Lemons, J. E. Biomaterials Science: An introduction to materials in medicine. (Academic Press, New York; 1996)). Another drawback of synthetic implants is the length of post-surgical rehabilitation. For dental implants, patients currently wait for several months following the placement of implant fixtures in the jaw bone, prior to the connection of dental prosthesis and functioning (Sumner, D. R., Turner, T. M. & Urban, R. M. Animal models relevant to cementless joint replacement. J. Musculoskelet. Neuronal. Interact. 1, 333-345 (2001)). Upon implant failure, revision surgeries are costly and technically challenging. Thus, strategies that enhance tissue ingrowth and long-term biofixation are critically needed.

Several approaches have been devised to improve tissue ingrowth in synthetic implants. Surface modification by changing topography or adsorbing bioactive cues is the most prevalent approach. Certain topographical features fabricated on implant surfaces are generally associated with enhanced cell adhesion, such as osteoblast adhesion to implant surface (McKoy, B. E., An, Y. H. & Friedman, R. J. in Mechanical Testing of Bone and the Bone-implant Interface. (eds. Y. H. An & R. A. Draughn) 439-462 (CRC Press, New York; 2000); Ingham, E. & Fisher, J. Biological reactions to wear debris in total joint replacement. Proc. Inst. Mech. Eng. [H]. 214, 21-37 (2000); Albrektsson, T., Branemark, P. I., Hansson, H. A. & Lindstrom, J. Osseointegrated titanium implants: Requirements for ensuring a long-lasting, direct bone-to-implant anchorage in man. Acta Orthop. Scand. 52, 155-170 (1981)). Bioactive cues are typically adsorbed to biomaterials, such as hydroxyapatite or hydrogel polymers, that are coated on the implant's surface.

The transforming growth factor β superfamily have been the most commonly used bioactive cues, including TGFβs and bone morphogenetic proteins (BMPs) (Lossdorfer, S. et al. Microrough implant surface topographies increase osteogenesis by reducing osteoclast formation and activity. J Biomed Mater Res A 70, 361-369 (2004); Meredith, D. O., Riehle, M. O., Curtis, A. S. & Richards, R. G. Is surface chemical composition important for orthopaedic implant materials? J. Mater. Sci. Mater. Med. 18, 405-413 (2007)). TGFβ1 plays a major role in the modulation of the behavior of multiple cell lineages, such as fibroblasts and osteoblasts that are of relevance to wound healing and tissue regeneration (Nebe, J. G., Luethen, F., Lange, R. & Beck, U. Interface Interactions of Osteoblasts with Structured Titanium and the Correlation between Physicochemical Characteristics and Cell Biological Parameters. Macromol Biosci 7, 567-578 (2007)). TGFβ1 also upregulates molecules such as alkaline phosphatase, type I collagen, bone sialoprotein and osteocalcin that are critical to tissue integration on implant surface, especially bone ingrowth in the implant's bone integration (Roberts, A. B. in Skeletal Growth Factors. (ed. E. Canalis) 233-249 (Lippincott, Williams, and Wilkins, Philadelphia; 2000)). TGFβ1 is further efficacious in increasing the calcium content and the size of calcified nodules of primary osteoblasts. BMP2 immersed in calcium phosphate-coated Ti implants yields approximately 50% more bone ingrowth (Alliston, T. N. & Derynck, R. in Skeletal Growth Factors. (ed. E. Canalis) 233-249 (Lippincott, Williams, and Wilkins, Philadelphia; 2000)). When adsorbed directly on Ti surface, BMP2 is not osteogenic, but BMP2 adsorbed in calcium phosphate coating on Ti surface induces bone ingrowth (Dimitriou, R., Tsiridis, E. & Giannoudis, P. V. Current concepts of molecular aspects of bone healing. Injury 36, 1392-1404 (2005)). Similarly, BMP7/OP1 adsorbed in peri-apatite coated Ti implant increases bone ingrowth by about 65% (Zhang, H., Aronow, M. S. & Gronowicz, G. A. Transforming growth factor-beta 1 (TGF-beta 1) prevents the age-dependent decrease in bone formation in human osteoblast/implant cultures. J Biomed Mater Res A 75, 98-105 (2005)). However, a critical drawback in this common approach of growth-factor adsorption is premature denaturation and diffusion of the delivered proteins or peptides, often within minutes of exposure to in vivo enzymes and catalysts (Liu, Y., Enggist, L., Kuffer, A. F., Buser, D. & Hunziker, E. B. The influence of BMP-2 and its mode of delivery on the osteoconductivity of implant surfaces during the early phase of osseointegration. Biomaterials 28, 2677-2686 (2007); Sumner, D. R., Turner, T. M., Urban, R. M., Virdi, A. S. & Inoue, N. Additive enhancement of implant fixation following combined treatment with rhTGF-beta2 and rhBMP-2 in a canine model. J. Bone Joint Surg. Am. 88, 806-817 (2006); Zhang, R. et al. Osteogenic protein-1 enhances osseointegration of titanium implants coated with peri-apatite in rabbit femoral defect. J. Biomed. Mater. Res. B Appl. Biomater. 71, 408-413 (2004)). Although the efficacy of cytokines adsorbed in implant-coating materials has been reported in animal models, higher cytokine doses are likely needed in humans, leading to high cost, potential toxicity and other obstacles in the regulatory process.

### SUMMARY OF THE INVENTION

Encapsulated bioactive cues are loaded into the pores of a porous implant device. The device can be made of a metal such as, for example, titanium. The cues can be encapsulated, for example inside microspheres (MPs). The bioactive cues thus encapsulated can be made bioactive in a controlled-release manner Suitable bioactive cues include activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin, fibronectin receptor α₅β₁, Factor X, HB-EGF, HBNF, HGF, HUAF, heart derived inhibitor of vascular cell proliferation, IFN-gamma, IL1 IGF-2 IFN-gamma, integrin receptors (e.g., various combinations of a subunits (e.g., α₁, α₂, α₃, α₄, ═₅, ═₆, α₇, α₈, α₉, α_{E}, αᵥ, α_{IIb}, α_{L}, α_{M}, α_{X}) and β subunits (e.g., β₁, β₂, β₃, β⁴, β₅, β₆, β₇, and β₈)), K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR.gamma , PPARγ ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-β, Tie 1, Tie2, TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF₁₆₄, VEGI, EG-VEGF, VEGF receptors, PF4, 16 kDa fragment of prolactin, prostaglandins E1 and E2, steroids, heparin, 1-butyryl glycerol (monobutyrin), and nicotinic amide. include transforming growth factor-beta 1 (TGFβ1). The encapsulating material is any suitable composition, such as poly-d-1-lactic-co-glycolic acid (PLGA). Loading is performed with procedures common to the art.

The controlled-release bioavailability profile increases the efficiency of bioactive cue uptake by the subject, resulting in effective treatment at greatly-reduced bioactive cue dosages, for example a ten-fold reduction in dosage. In some embodiments the implant device is hollow, and within the hollow cavity is placed a matrix in which encapsulated biocues are loaded. The size of the pores on the surface of the device are chosen to selectively alter the controlled-release bioavailability profile of the cue.

In some embodiments of the invention the implant device is made from metals other than titanium, such as stainless steel, titanium-based alloys (eg. Ti—Al—V alloys and Ti—Al—Nb alloys) and cobalt-chromium based alloys.

In some embodiments of the invention the average diameter of the encapsulating MP is 100+70 μm.

In some embodiments of the invention the invention can comprise a porous implantable medical device comprising a device body, a plurality of pores contacting a surface of said device; and at least one encapsulated bioactive cue within at least one of said pores. Other embodiments comprise a plurality of pores contacting a surface of the device, wherein at least some of said pores are interconnected such that some of said interconnected pores form throughbores which connect said device's inner and outer surfaces; and at least one encapsulated bioactive cue within at least one of said pores. In other embodiments, the bioactive cue is made bioavailable in a controlled-release manner. In other embodiments, the pores of the device are of a non-uniform size.

In other embodiments, the device is at least partially hollow.

In some embodiments, the encapsulating material is chosen from a material selected from the group consisting of polylactic acid (PLA), polyglycolid acid (PGA), copolymers of lactic acid and glycolic acid (PLGA), polycaprolactone, polyphosphoester, polyorthoester, poly(hydroxy butyrate), poly(diaxanone), poly(hydroxy valerate), poly(hydroxy butyrate-co-valerate), poly(glycolide-co-trimethylene carbonate), polyanhydrides, polyphosphoester, poly(ester-amide), polyphosphoeser, polyphosphazene, poly(phosphoester-urethane), poly(amino acids), polycyanoacrylates, biopolymeric molecules such as fibrin, fibrinogen, cellulose, starch, collagen and hyaluronic acid, and mixtures and copolymers of the foregoing.

In some embodiments, the bioactive cue is selected from the group consisting of a growth factor, a cytokine, DNA, RNA, a transcription factor, a tissue ingrowth modulator, and a tissue adhesion modulator. In some embodiments, the device has a plurality of different cues

In other embodiments, the invention is an aspect of a method of preparing a porous, implantable medical device comprising providing a porous, implantable medical device with a plurality of pores contacting a surface of the device, encapsulating at least one bioactive cue; and adding the encapsulated bioactive cue within at least one of said pores.

Other embodiments, the invention is an aspect of a method for treating a subject, comprising diagnosing the subject's affliction; and determining the appropriate agent to administer to the subject; and preparing an implantable device with the appropriate agent by the method of claim 7; and implanting the device in the subject.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 shows a sample of poly-d-1-lactic-co-glycolic acid (PLGA) MPs fabricated by double emulsion under light microscopy, with an average diameter of 64±16 μm (FIG. 1a), which can be fine tuned for yielding different release kinetics, as well as dose comparisons and release profiles for the different delivery systems.

FIG. 2 shows a hollow Ti implant with microencapsulated TGFβ1 or placebo MPs placed in hMSC culture. Microparticles were observed inside the hollow Ti implant up to the tested 28 days. Adjacent to the outer wall of the hollow Ti implant, abundant hMSC accumulated in response to control-released 1 ng/mL TGFβ1 at 28 days.

FIG. 3 shows the device implanted within the leg of a rabbit, as well as photographs of experiment results comparing the effects of MP-delivered TGFβ1 and adsorbed TGFβ1.

FIG. 4 shows ingrowth of substantial woven bone (WB) that was integrated with cortical bone (CB) for both 1 ng gelatin-adsorbed TGFβ1 implant and 1 ng/mL control-released TGFβ1 implant, in comparison to moderate WB formation in the TGFβ1-free implant.

FIG. 5 shows Scanning electron microscopy (SEM) of marked bone-to-implant contact (BIC) and WB formation in the surface and pores of both 1 ng gelatin-adsorbed TGFβ1 implant and 1 ng/mL control-released TGFβ1 implant, in comparison with the TGFβ1-free or placebo MP implant.

### DETAILED DESCRIPTION OF THE INVENTION

A controlled-release system overcomes the limitations of rapid denaturation and diffusion of growth factors in vivo, thus reducing drug dose. An effective controlled-release system is achieved by encapsulating bioactive cues in biocompatible and biodegradable microparticles (Liu, Y., Enggist, L., Kuffer, A. F., Buser, D. & Hunziker, E. B. The influence of BMP-2 and its mode of delivery on the osteoconductivity of implant surfaces during the early phase of osseointegration. Biomaterials 28, 2677-2686 (2007); Sumner, D. R., Turner, T. M., Urban, R. M., Virdi, A. S. & Inoue, N. Additive enhancement of implant fixation following combined treatment with rhTGF-beta2 and rhBMP-2 in a canine model. J. Bone Joint Surg. Am. 88, 806-817 (2006); Zhang, R. et al. Osteogenic protein-1 enhances osseointegration of titanium implants coated with peri-apatite in rabbit femoral defect. J. Biomed. Mater. Res. B Appl. Biomater. 71, 408-413 (2004)). For the replacement of diseased or missing tissue, synthetic implants are advantageous because they eliminate donor site morbidity, they have a virtually endless supply, and they have a potential for packaged delivery in the operating room. In contrast to current solid and inert prosthesis design, the present invention provides porous implants that serve as delivery framework for controlled release of microencapsulated bioactive cues.

The biocompatible and biodegradable encapsulating material of the present invention can be either a homopolymer, a copolymer, or a polymer blend that is capable of releasing the pharmacologically active agent into at least one target site in the arterial walls in a controlled and sustained manner after local injection. Suitable polymeric materials that can be used in the present invention include, but are not limited to: polylactic acid (PLA), polyglycolid acid (PGA), copolymers of lactic acid and glycolic acid (PLGA), polycaprolactone, polyphosphoester, polyorthoester, poly(hydroxy butyrate), poly(diaxanone), poly(hydroxy valerate), poly(hydroxy butyrate-co-valerate), poly(glycolide-co-trimethylene carbonate), polyanhydrides, polyphosphoester, poly(ester-amide), polyphosphoeser, polyphosphazene, poly(phosphoester-urethane), poly(amino acids), polycyanoacrylates, biopolymeric molecules such as fibrin, fibrinogen, cellulose, starch, collagen and hyaluronic acid, and mixtures and copolymers of the foregoing.

Preferably, the biocompatible and biodegradable polymeric material of the microparticles (MPs) is selected from the group consisting of PLA, PGA, PLGA, and mixtures thereof More preferably, the biocompatible and biodegradable polymeric material of-the present invention comprises the PLGA copolymer. The PLA, PGA, or PLGA polymers can be any of D-, L- and D-/L-configuration. PLGA microspheres can be readily tailored towards specific degradation needs by modifying the ratio of PLA:PGA. The methyl group in PLA is responsible for its hydrophobic and slow degradation. PGA is crystalline and increases degradation times. Therefore, different ratios of PGA and PLA accommodate specific growth factor release rates.

As the MPs degrade, bioactive cues within the pores of the device are released over time via pre-designed release profiles. Microparticle-encapsulated and controlled-release TGFβ3 at up to 1 ng/mL inhibits the osteogenic differentiation of bone marrow-derived human mesenchymal stem cells (hMSC) and the elaboration of an osteogenic matrix (Sumner, D. R., Turner, T. M., Urban, R. M., Virdi, A. S. & Inoue, N. Additive enhancement of implant fixation following combined treatment with rhTGF-beta2 and rhBMP-2 in a canine model. J. Bone Joint Surg. Am. 88, 806-817 (2006); Moioli, E. K., Clark, P. A., Xin, X., Lal, S. & Mao, J. J. Matrices and scaffolds for drug delivery in dental, oral and craniofacial tissue engineering. Adv Drug Deliv Rev (2007)), presenting potential applications in wound healing including the inhibition of ectopic bone formation. The recruitment and proliferation of MSC enriches the populations of osteoprogenitors and osteoblasts, and are critical to the initial stage of implant wound healing (Sumner, D. R., Turner, T. M., Urban, R. M., Virdi, A. S. & Inoue, N. Additive enhancement of implant fixation following combined treatment with rhTGF-beta2 and rhBMP-2 in a canine model. J Bone Joint Surg. Am. 88, 806-817 (2006); Moioli, E. K., Clark, P. A., Xin, X., Lal, S. & Mao, J. J. Matrices and scaffolds for drug delivery in dental, oral and craniofacial tissue engineering. Adv Drug Deliv Rev (2007)). Porous implant surfaces provide a further mechanism for selectively controlling the time-release bioavailability profile by partially shielding the encapsulated bioactive cues from bioavailability. Here, a porous titanium implant is fabricated for the delivery of microencapsulated bioactive cues. Together, these features provide for controlled release of bioactive cues. Controlled-release TGFβ1 promotes the proliferation and migration of human mesenchymal stem cells into porous implants in vitro. Upon 4-wk implantation in the rabbit humerus, controlled-release TGFβ1 from porous implants significantly increased BIC by 96% and bone ingrowth by 50% over placebos. Controlled-release 100 ng TGFβ1 induced equivalent BIC and bone ingrowth to adsorbed 1 μg TGFβ1, suggesting that controlled release is effective at 10-fold less drug dose than adsorption. The present observation of increased bone-to-implant contact by 96% and bone ingrowth by 50% via control-released TGFβ1 over placebo microparticles is comparable to a number of reported in vivo studies of the efficacy of bone ingrowth by growth-factor adsorption in biomaterials that coat implant surface (Moioli, E. K., Hong, L. & Mao, J. J. Inhibition of osteogenic differentiation of human mesenchymal stem cells. Wound Repair Regen. 15, 413-424 (2007); Alhadlaq, A. et al. Adult stem cell driven genesis of human-shaped articular condyle. Ann. Biomed. Eng. 32, 911-923 (2004)), but with the important difference that the present controlled-release approach reduces drug dose by 10 fold. This 10-fold decrease in drug dose can have significant implications in potential reduction in the cost and toxicity of in vivo delivered biological cues. Present findings further provide strong evidence that control-released TGFβ1 via porous Ti implants not only induces the migration and proliferation of hMSC in vitro, but also enhances bone ingrowth and bone-to-implant contact in vivo. Thus, the excessive mass of solid implants can be made porous as a drug delivery carrier for controlled-release of microencapsulated bioactive cues. Porous implant design also increases the surface area for cell adhesion and bone ingrowth. New bone growing into the interconnecting pores of porous implants, as shown in the present study, can provide bone interlocking, further enhancing bone ingrowth and long-term periprosthetic bone health.

The present invention can incorporate a number of different bioactive cues. Non-limiting examples of bioactive cues include activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin, fibronectin receptor α₅β₁, Factor X, HB-EGF, HBNF, HGF, HUAF, heart derived inhibitor of vascular cell proliferation, IFN-gamma, ILL IGF-2 IFN-gamma, integrin receptors (e.g., various combinations of a subunits (e.g., α₁, α₂, α₃, α₄, α₅, α₆, α₇, α₈, α₉, α_{E}, α_{V}, α_{IIb}, α_{L}, α_{M}, α_{X}) and β subunits (e.g., β₁, β₂, β₃, β₄, β₅, β₆, β₇, and β₈)), K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR.gamma , PPARy ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-β, Tie 1, Tie2, TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF₁₆₄, VEGI, EG-VEGF, VEGF receptors, PF4, 16 kDa fragment of prolactin, prostaglandins E1 and E2, steroids, heparin, 1-butyryl glycerol (monobutyrin), and nicotinic amide.

In some embodiments, the bioactive cues include tissue progenitor cells. For example, the tissue progenitor cell can be a mesenchymal stem cell (MSC), MSC-derived cell, osteoblast, chondrocyte, myocyte, adipocyte, neuronal cell, neuronal supporting cells such as Schwann cells, neural glial cells, fibroblastic cells including interstitial fibroblasts, tendon fibroblasts or tenocytes, ligament fibroblasts, periodontal fibroblasts, craniofacial fibroblasts, gingival fibroblasts, periodontal fibroblasts, cardiomyocytes, epithelial cells, dermal fibroblasts, liver cells, uretheral cells, kidney cells, periosteal cells, bladder cells, or beta-pancreatic islet cell. For example, when engineering vascularized bone tissue, the tissue progenitor cells infused into the matrix material can be selected from mesenchymal stem cells (MSC), MSC-derived osteoblasts, MSC-derived chondrocytes, or other similar progenitor cells that can give rise to bone cells. As another example, when engineering vascularized adipose tissue, the tissue progenitor cells infused into the matrix material can be selected from MSCs, MSC-derived adipogenic cells, or other similar progenitor cells that can give rise to adipose cells.

In some embodiments, the bioactive cues include vascular progenitor cells. Vascular progenitor cells include, for example, hematopoietic stem cells (HSC), HSC-derived endothelial cells, blood vascular endothelial cells, lymph vascular endothelial cells, endothelial cell lines, primary culture endothelial cells, endothelial cells derived from stem cells, bone marrow derived stem cells, cord blood derived cells, human umbilical vein endothelial cells (HUVEC), lymphatic endothelial cells, endothelial pregenitor cells, and stem cells that differentiate into endothelial cells, endothelial cell lines, endothelial cells generated from stem cells in vitro, endothelial cells from adipose tissue, smooth muscle cells, interstitial fibroblasts, myofibroblasts, periodontal tissue or tooth pulp, and vascular derived cells, or other similar progenitor cells that can give rise to vascular cells.

In some embodiments, a matrix is placed within the implant device. The matrix material can be seeded with one or more cell types in addition to a first tissue progenitor cell and a first vascular progenitor cell. Such additional cell type can be selected from those discussed above, and/or can include (but not limited to) skin cells, liver cells, heart cells, kidney cells, pancreatic cells, lung cells, bladder cells, stomach cells, intestinal cells, cells of the urogenital tract, breast cells, skeletal muscle cells, skin cells, bone cells, cartilage cells, keratinocytes, hepatocytes, gastro-intestinal cells, epithelial cells, endothelial cells, mammary cells, skeletal muscle cells, smooth muscle cells, parenchymal cells, osteoclasts, or chondrocytes. These cell-types can be introduced prior to, during, or after vascularization of the seeded matrix. Such introduction can take place in vitro or in vivo. When the cells are introduced in vivo, the introduction can be at the site of the engineered vascularized tissue or organ composition or at a site removed therefrom. Cells implanted in this manner can stimulate bone growth from within and through the device. Exemplary routes of administration of the cells include injection and surgical implantation

In some embodiments, the progenitor cells used to seed the matrix are transformed with a heterologous nucleic acid so as to express a bioactive molecule, or heterologous protein or to overexpress an endogenous protein. As an example, the progenitor cells to be seeded in the matrix can be genetically modified to expresses a fluorescent protein marker. Exemplary markers include GFP, EGFP, BFP, CFP, YFP, and RFP. As another example, progenitor cells to be seeded in the matrix can be genetically modified to express an angiogenesis-related factor, such as activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin and fibronectin receptor α₅β₁, Factor X, HB-EGF, HBNF, HGF, HUAF, heart derived inhibitor of vascular cell proliferation, IFN-gamma, ILL IGF-2 IFN-gamma, integrin receptors (e.g., various combinations of a subunits (e.g., α₁, α₂, α₃, α₄, α₅, α₆, α₇, α₈, α₉, α_{E}, α_{V}, α_{IIb}, α_{L}, α_{M}, α_{X}), K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR.gamma., PPAR.gamma. ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-beta, Tie 1, Tie2, TGF-β, and TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF.sub.164, VEGI, EG-VEGF, VEGF receptors, PF4, 16 kDa fragment of prolactin, prostaglandins E1 and E2, steroids, heparin, 1-butyryl glycerol (monobutyrin), or nicotinic amide. As another example, progenitor cells to be seeded in the matrix can be transfected with genetic sequences that are capable of reducing or eliminating an immune response in the host (e.g., expression of cell surface antigens such as class I and class II histocompatibility antigens can be suppressed). This can allow the transplanted cells to have reduced chance of rejection by the host. Suitable immunosuppressive agents that can be administered include, but are not limited to, steroids, cyclosporine, cyclosporine analogs, cyclophosphamide, methylprednisone, prednisone, azathioprine, FK-506, 15-deoxyspergualin, and other immunosuppressive agents that act by suppressing the function of responding T cells. Other immunosuppressive agents that can be administered in combination with the growth factor formulations include, but are not limited to, prednisolone, methotrexate, thalidomide, methoxsalen, rapamycin, leflunomide, mizoribine (bredinin™), brequinar, deoxyspergualin, and azaspirane (SKF 105685), Orthoclone OKT™ 3 (muromonab-CD3). Sandimmune™, Neoral™, Sangdya™ (cyclosporine), Prograf™ (FK506, tacrolimus), Cellcept™ (mycophenolate motefil, of which the active metabolite is mycophenolic acid), Imuran™ (azathioprine), glucocorticosteroids, adrenocortical steroids such as Deltasone™ (prednisone) and Hydeltrasol™ (prednisolone), Folex™ and Mexate™ (methotrxate), Oxsoralen-Ultra™ (methoxsalen) and Rapamuen™ (sirolimus).

In some embodiments, the bioactive cues include a chemotherapeutic agent or immunomodulatory molecule. Such agents and molecules are known to the skilled artisan. Transforming growth factor-beta 1 (TGFβ1) and BMP2 are both efficacious in enhancing implant bone ingrowth (Alliston, T. N. & Derynck, R. in Skeletal Growth Factors. (ed. E. Canalis) 233-249 (Lippincott, Williams, and Wilkins, Philadelphia; 2000)); Zhang, H., Aronow, M. S. & Gronowicz, G. A.

TGFβ1 prevents age-dependent decrease in bone formation in human osteoblast/implant cultures. J Biomed Mater Res A 75, 98-105 (2005)). TGFβ1 stimulates the production of fibronectin, collagen, integrin and proteoglycans (Wang, X. & Mao, J. J. Accelerated chondrogenesis of the rabbit cranial base growth plate by oscillatory mechanical stimuli. J. Bone Miner. Res. 17, 1843-1850 (2002); Kopher, R. A. & Mao, J. J. Suture growth modulated by the oscillatory component of micromechanical strain. J. Bone Miner. Res. 18, 521-528 (2003); Clark, P. A., Rodriguez, A., Sumner, D. R., Hussain, M. A. & Mao, J. J. Modulation of bone ingrowth of rabbit femur titanium implants by in vivo axial micromechanical loading. J. Appl. Physiol. 98, 1922-1929 (2005)). In contrast, control-released TGFβ1 at the same dose, 100 ng, significantly augments bone ingrowth. Although a higher dose of adsorbed 1 μg gelatin-adsorbed TGFβ1 was as effective as 100 ng control-released TGFβ1, the proportionally high dose in association with adsorption in patients can present as problems such as toxicity, high cost and regulatory difficulties.

Because dental and orthopedic implants are inserted into endosteal bone, the role played by multipotent bone marrow-derived (De Ranieri, A. et al. Local application of rhTGF-beta2 enhances peri-implant bone volume and bone-implant contact in a rat model. Bone 37, 55-62 (2005); Lind, M. Growth factor stimulation of bone healing. Effects on osteoblasts, osteomies, and implants fixation. Acta Orthop. Scand. Suppl. 283, 2-37 (1998)) warrant investigations. However, previous implant studies examined the participation of osteoblasts in implant wound healing, but rarely MSC 12 (Meredith, D. O., Riehle, M. O., Curtis, A. S. & Richards, R. G. Is surface chemical composition important for orthopaedic implant materials? J. Mater. Sci. Mater. Med. 18, 405-413 (2007); Nebe, J. G., Luethen, F., Lange, R. & Beck, U. Interface Interactions of Osteoblasts with Structured Titanium and the Correlation between Physicochemical Characteristics and Cell Biological Parameters. Macromol Biosci 7, 567-578 (2007); Alhadlaq, A. et al. Adult stem cell driven genesis of human-shaped articular condyle. Ann. Biomed. Eng. 32, 911-923 (2004); Schliephake, H. et al. Functionalization of dental implant surfaces using adhesion molecules. J Biomed Mater Res B Appl Biomater 73, 88-96 (2005); Linkhart, T. A., Mohan, S. & Baylink, D. J. Growth factors for bone growth and repair: IGF, TGF beta and BMP. Bone 19, 1S-12S (1996)). The presently observed chemotaxis and proliferation of MSC likely enrich the populations of osteoprogenitors and osteoblasts that are critical to implant wound healing. Thus, the augmentation of bone ingrowth in the surface and pores of Ti implants in vivo is likely contributed by the modulation of the chemotaxis and proliferation of MSC by control-released TGFβ1. Although TGFβ1 can conceptually have attracted cell lineages other than MSC or osteoblasts, integration of the rabbit humerus implants, stability upon harvest and peri-implant bone formation provide evidence against the sum effects of overwhelming attachment of, for example, fibroblasts, to implant surface. Besides orthopedic and dental prostheses, other applications of porous implants can include spinal cages, coronary implants, maxillofacial implants or any solid prostheses in current use but without the delivery of bioactive cues, especially by controlled release. The present approach relies on the homing of host cells that are involved in implant bone healing, offering an attractive modality for translation. Transformation of inert and solid synthetic implants into porous, bioactive drug delivery systems accelerate tissue integration in the restoration of the function of diseased or missing tissues and organ.

### EXAMPLES

The following non-limiting examples are provided to further illustrate the present invention. It will be appreciated by those of skill in the art that the techniques disclosed in the examples that follow represent approaches the inventors have found function well in the practice of the invention, and thus can be considered to constitute examples of modes for its practice. However, those of skill in the art will, in light of the present disclosure, appreciate that many changes can be made in the specific embodiments that are disclosed and still obtain a like or similar result without departing from the spirit and scope of the invention.

### Example 1

### Dose-Independent Release Kinetics of Microencapsulated TGFβ1

### Microencapsulation OF TGFβ1

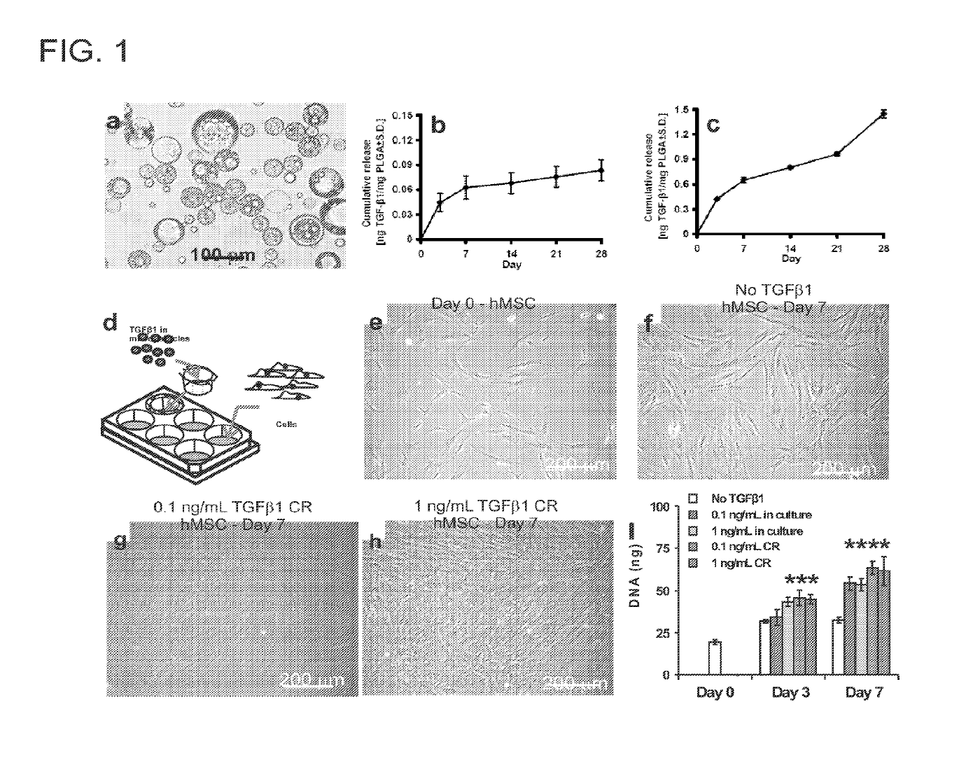

Microencapsulation of transforming growth factor β1 (TGFβ1) in poly-lactic-co-glycolic acid (PLGA) (FIG. 1a) was achieved using a double emulsion technique ([water-in-oil]-in-water) (Sumner, D. R., Turner, T. M., Urban, R. M., Virdi, A. S. & Inoue, N. Additive enhancement of implant fixation following combined treatment with rhTGF-beta2 and rhBMP-2 in a canine model. J Bone Joint Surg. Am. 88, 806-817 (2006)). Recombinant human TGFβ1 with a molecular weight of 25 kDa (R&D Systems, Minneapolis, Minn.) was reconstituted in 1% bovine serum albumin (BSA) solution. MPs were observed using a light microscope, with their average diameter measured by fitting circles to match randomly selected microparticles. The MPs were frozen in liquid nitrogen, lyophilized (Sumner, D. R. et al. Enhancement of bone ingrowth by transforming growth factor-beta. J Bone Joint Surg. Am. 77, 1135-1147 (1995)) freeze-dried, and stored at −20° C. Placebo MPs encapsulating PBS were used as controls to determine any potential effects of PLGA degradation byproducts (Sumner, D. R., Turner, T. M., Urban, R. M., Virdi, A. S. & Inoue, N. Additive enhancement of implant fixation following combined treatment with rhTGF-beta2 and rhBMP-2 in a canine model. J Bone Joint Surg. Am. 88, 806-817 (2006)). This primary emulsion was vortexed and stabilized in 1% polyvinyl alcohol (PVA, 30,000-70,000 MW, Sigma, St. Louis, Mo.) ([water-in-oil]-in-water). The resulting mixture was added to 100 mL of 0.1% PVA solution for 1 min, followed by the addition of 100 mL of 2% isopropanol, and is stirred under a fume hood for 2 hrs at 400-500 rpm to allow for solvent vaporization solvent (dichloromethane). The MPs were collected by filtration through a 2 μm filter. Initial encapsulation efficiency was determined by dissolving 10 mg of TGFβ1-encapsulated MPs in dichloromethane, adding 1% BSA, and allowing the solution to separate overnight. The released TGFβ1 from MPs was quantified from the aqueous phase using an enzyme linked immunosorbent assay (ELISA), with its encapsulation efficiency calculated as previously described (Sumner, D. R. et al. Enhancement of bone ingrowth by transforming growth factor-beta. J. Bone Joint Surg. Am. 77, 1135-1147 (1995).

### TGFβ1 Release Kinetics

The release kinetics of TGFβ1 from the PLGA microparticles was determined and used to calculate dosing in subsequent studies (FIGS. 1b,c). MPs were suspended in 1% BSA, set in a water bath at 37° C., shaken at 60 rpm, and centrifuged at 5000 rpm, followed by the collection of supernatant. The PLGA microparticles were then resuspended in 1% BSA and placed back in water bath. The total amount of TGFβ1 in each supernatant sample was quantified using ELISA to construct release kinetics (Sumner, D. R., Turner, T. M., Urban, R. M., Virdi, A. S. & Inoue, N. Additive enhancement of implant fixation following combined treatment with rhTGF-beta2 and rhBMP-2 in a canine model. J. Bone Joint Surg. Am. 88, 806-817 (2006); Sumner, D. R. et al. Enhancement of bone ingrowth by transforming growth factor-beta. J. Bone Joint Surg. Am. 77, 1135-1147 (1995)). A 50 μl solution containing either 250 ng of TGFβ1 (low dose as in FIG. 1b), 2.5 μg of TGFβ1 (high dose as in FIG. 1c), or phosphate buffered saline (PBS—placebo control) was added to a 25% w/v PLGA solution (Sigma, St. Louis, Mo.) that was dissolved in dichloromethane (250 mg PLGA:1 mL dichloromethane).

A sample of poly-d-1-lactic-co-glycolic acid (PLGA) MPs fabricated by double emulsion is shown under light microscopy, with an average diameter of 64±16 μm (FIG. 1a), which can be fine tuned for yielding different release kinetics. To determine whether the initial encapsulation dose affects release kinetics, a low dose of 250 ng TGFβ1 (FIG. 1b) was compared with and a high dose of 2.5 μg TGFβ1 (FIG. 1c), both encapsulated in 250 mg PLGA. The release profiles were similar regardless of the initial TGFβ1 encapsulation amount (FIG. 1b,c), suggesting the stability and versatility of the present drug delivery system. For both low and high TGFβ1 doses, an initial burst release at day 3 was sustained up to the tested 4 wks (FIG. 1b,c), consistent with previous demonstration of control-released growth factors in vitro up to several months (Sumner, D. R., Turner, T. M., Urban, R. M., Virdi, A. S. & Inoue, N. Additive enhancement of implant fixation following combined treatment with rhTGF-beta2 and rhBMP-2 in a canine model. J Bone Joint Surg. Am. 88, 806-817 (2006); Moioli, E. K., Clark, P. A., Xin, X., Lal, S. & Mao, J. J. Matrices and scaffolds for drug delivery in dental, oral and craniofacial tissue engineering. Adv Drug Deliv Rev (2007)). As anticipated, a 10-fold higher release dose was observed with 2.5 μg TGFβ1 (FIG. 1c) than with 250 ng TGFβ1 (FIG. 1b), further indicating the efficacy of the drug delivery system.

DATA analysis AND STATISTICS: An One-Way Analysis of Variance (ANOVA) with post-hoc Bonferroni tests was performed to determine any significant differences between or within all groups in which numerical data were generated using at an a level of p<0.05.

### Example 2

### Controlled-Release Tgfβ1 Induces The Proliferation of Human Mesenchymal Stem Cells in Monolayer Culture

### Isolation of Human Mesenchymal Stem Cells

Fresh bone marrow samples of multiple adult male donors (AllCells, Berkeley, Calif.) were used to isolate MSC. Non-adherent cells were removed by negative selection (Sumner, D. R., Turner, T. M., Urban, R. M., Virdi, A. S. & Inoue, N. Additive enhancement of implant fixation following combined treatment with rhTGF-beta2 and rhBMP-2 in a canine model. J Bone Joint Surg. Am. 88, 806-817 (2006); Moioli, E. K., Hong, L., Guardado, J., Clark, P. A. & Mao, J. J. Sustained release of TGFbeta3 from PLGA microspheres and its effect on early osteogenic differentiation of human mesenchymal stem cells. Tissue Eng. 12, 537-546 (2006)). Adherent cells were layered on a Ficoll-Paque gradient (StemCell Technologies), followed by the removal of the entire layer of enriched cells from Ficoll-Paque interface. The isolated mononuclear and adherent cells were counted under an inverted microscope, plated in basal medium (Dulbecco's Modified Eagle's Medium+10% fetal bovine serum+1% antibiotic-antimycotic) at approximately 0.5-1×10⁶ cells per 100-mm Petri dish, and incubated at 37° C. and 5% CO₂ After 24 hrs, non-adherent cells were discarded, and adherent cells were washed twice with PBS and incubated for 12 days with a medium change every 3 to 4 days. The remaining mononuclear and adherent cells consist of heterogeneous cell lineages including MSC (Moioli, E. K., Hong, L., Guardado, J., Clark, P. A. & Mao, J. J. Sustained release of TGFbeta3 from PLGA microspheres and its effect on early osteogenic differentiation of human mesenchymal stem cells. Tissue Eng. 12, 537-546 (2006)) Upon 80 to 90% confluence, primary MSC were trypsinized and passaged, approximately every 7 days.

### Cell Proliferation Assay

PLGA microparticles were sterilized by ethylene oxide (EO), which does not significantly affect the release profile (Sumner, D. R., Turner, T. M., Urban, R. M., Virdi, A. S. & Inoue, N. Additive enhancement of implant fixation following combined treatment with rhTGF-beta2 and rhBMP-2 in a canine model. J. Bone Joint Surg. Am. 88, 806-817 (2006)). The release profile of TGFβ1 showed the release of 0.06 ng/mg TGFβ1 after 7 days by culturing with 5 mg or 50 mg of MPs and 3 mL of growth medium, a solution concentration of 0.1 ng/mL or 1 ng/ml, respectively, of TGFβ1. Either 5 or 50 mg of MPs (low density), corresponding to 0.1 or 1 ng/mL of TGFβ1 released after 7 days, respectively, was placed in a transwell insert with an 0.4 μm dia. porous membrane (FIG. 1d). Transwell inserts allowed MPs to be suspended 0.9 mm above a monolayer of hMSC, while the pores allowed passage of TGFβ1 released from the PLGA microparticles (Sumner, D. R., Turner, T. M., Urban, R. M., Virdi, A. S. & Inoue, N. Additive enhancement of implant fixation following combined treatment with rhTGF-beta2 and rhBMP-2 in a canine model. J. Bone Joint Surg. Am. 88, 806-817 (2006)), (FIG. 1d). Five milligrams of MPs encapsulating PBS were used as placebo controls. For hMSC exposed to TGFβ1 in solution, the TGFβ1 was diluted to the desired concentration in corresponding medium and replenished every media change. The transwell inserts containing PLGA microparticles were placed into the 6-well dishes over the monolayers of hMSC and cultured for 0, 3, and 7 days (FIG. 1e-h). Medium was changed at day 5 to maximize the bioactivity of control-released TGFβ1 from PLGA microparticles. At each time point, corresponding monolayers of cells were submersed in 0.5 mL of 1% Triton-X for 20 min, collected using a cell scraper, and homogenized using sonification to form a cell lysate. Total DNA content of the cell lysate was determined using Hoechst 33258 dye (Fluorescent DNA Quant. Kit; BioRad; Hercules, Calif.), per prior methods (Sumner, D. R., Turner, T. M., Urban, R. M., Virdi, A. S. & Inoue, N. Additive enhancement of implant fixation following combined treatment with rhTGF-beta2 and rhBMP-2 in a canine model. J. Bone Joint Surg. Am. 88, 806-817 (2006)), (FIG. 1i). The bioactivity of control-released TGFβ1 was tested using a proliferation assay. Various concentrations of control-released TGFβ1 were compared with dose-corresponding TGFβ1 added in cell culture (without microencapsulation (FIG. 1i).

Fresh bone marrow samples of multiple adult male donors were prepared to isolate hMSC, per previous approaches (Moioli, E. K., Clark, P. A., Xin, X., Lal, S. & Mao, J. J. Matrices and scaffolds for drug delivery in dental, oral and craniofacial tissue engineering. Adv Drug Deliv Rev (2007); Holland, T. A. et al. Degradable hydrogel scaffolds for in vivo delivery of single and dual growth factors in cartilage repair. Osteoarthritis Cartilage 15, 187-197 (2007); Moioli, E. K., Hong, L., Guardado, J., Clark, P. A. & Mao, J. J. Sustained release of TGFbeta3 from PLGA microspheres and its effect on early osteogenic differentiation of human mesenchymal stem cells. Tissue Eng. 12, 537-546 (2006)). The effects of control-released TGFβ1 on the proliferation rates of hMSC were compared with dose-matched TGFβ1 added to culture medium (without microencapsulation). A submerged transwell system allowed the release of microencapsulated TGFβ1 into the underlying cells in culture medium, and yet without direct contact between MPs and cells (FIG. 1d). Microscopically, marked hMSC proliferation at day 7 was observed with control-released TGFβ1 at 0.1 ng/mL (FIG. 1g), and more markedly at 1 ng/mL (FIG. 1h), in comparison to day 0 (FIG. 1e) or no TGFβ1 delivery at day 7 (FIG. 1f). These qualitative observations of cell proliferation are substantiated quantitatively by DNA content of hMSC. When treated with control-released TGFβ1 at either 0.1 ng/mL (n=6, p<0.01) or 1 ng/mL (n=6, p<0.01 at day 3, p<0.05 at day 7), DNA content was significantly higher than placebo MPs at days 3 and 7 (FIG. 1i). Importantly, the DNA content of hMSC treated with control-released TGFβ1 at either 0.1 or 1 ng/mL showed no significant differences from that of dose-matched TGFβ1 added to culture medium (FIG. 1i), further indicating the efficacy of the controlled-release system.

### Example 3

### Controlled-Release TGFβ1 From Hollow Titanium Implant is Chemotactic to Human Mesenchymal Stem Cells

### Three Dimensional (3D) In Vitro Cell Migration Model

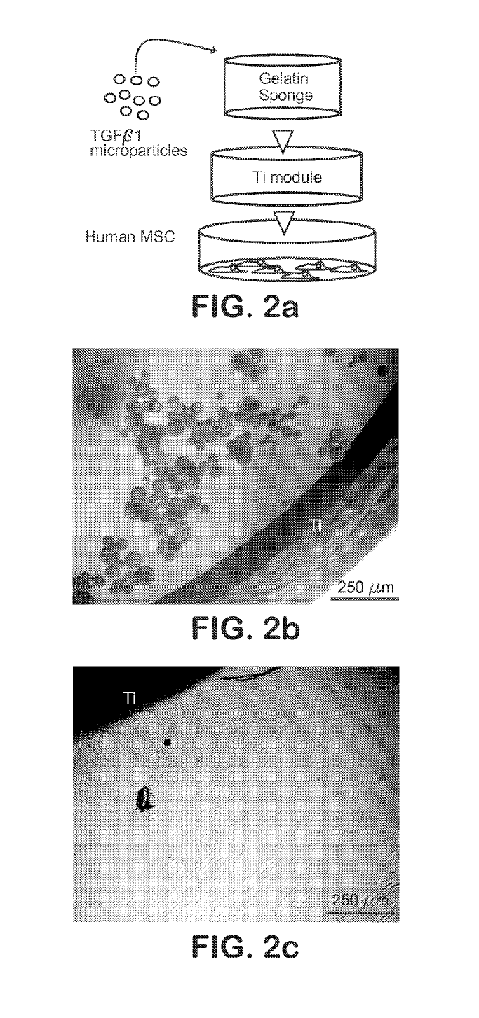

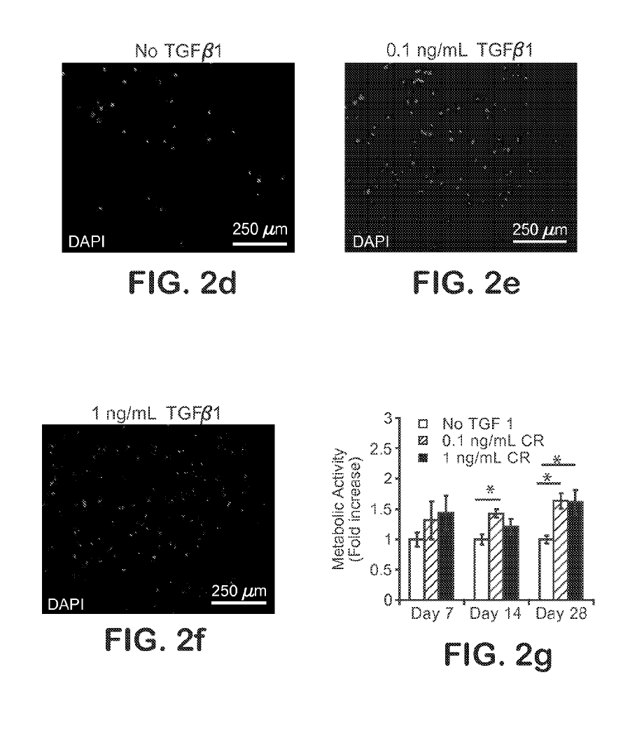

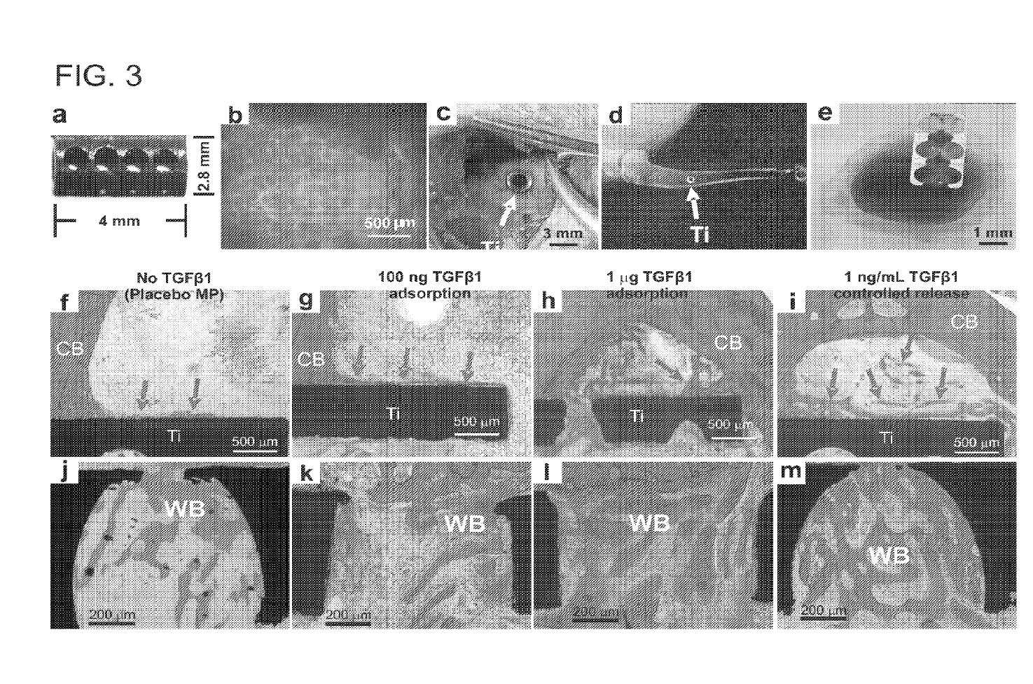

A gelatin sponge (Gelfoam, Pharmacia, Kalamazoo, Mich.) with pore sizes of 200-500 μm was chosen, given its previously demonstrated support of hMSC growth and wide use in bone regeneration (Cohen, S., Yoshioka, T., Lucarelli, M., Hwang, L. H. and Langer, R. Controlled delivery systems for proteins based on poly(lactic/glycolic acid) microspheres. Pharm. Res. 8, 713-720 (1991)). Scanning electron microscopy (SEM) (Hitachi, S-3000N) confirmed the pore size range of 200-500 μm (FIG. 3b). A hollow Ti implant module (7×6 mm; 1.×dia.) was fabricated and sterilized by autoclave (FIG. 2a). MPs encapsulating TGFβ1 or PBS (placebo control) were infused into the gelatin sponge by negative pressure, which was inserted in the hollow core of the Ti implant (FIG. 2a). The hollow Ti implant was placed in a monolayer of hMSC (FIG. 2a). The following TGFβ1 doses and delivery modes were investigated: 5 mg of low-density TGFβ1 MPs (≈0.1 ng/mL TGFβ1), 5 mg of high-density TGFβ1 MPs (≈1 ng/mL TGFβ1), or 5 mg of placebo MPs encapsulating PBS. Cell culture was incubated with fresh medium changes every 5 days. At pre-designated 7, 14, and 28 days, gelatin sponges from inside the Ti implants were removed and rinsed. Cell metabolic activities were determined using a colorimetric assay with a tetrazolium compound [3-(4,5-dimethylthiazol-2-yl)-5-(3-carboxymethoxyphenyl)-2-(4-sulfophenyl)-2H-tetrazolium, inner salt; MTS] following manufacturer's protocol (CellTiter 96 AQueous One Solution Cell Proliferation Assay, Promega, Madison, Wisc.) and per prior methods (Sumner, D. R., Turner, T. M., Urban, R. M., Virdi, A. S. & Inoue, N. Additive enhancement of implant fixation following combined treatment with rhTGF-beta2 and rhBMP-2 in a canine model. J. Bone Joint Surg. Am. 88, 806-817 (2006)). The MTS solution was diluted 1:10 in serum-free DMEM without phenol red. Each gelatin sponge was completely immersed in MTS and incubated for 1 hr, followed by the collection of the supernatant and reading on a microplate reader at 490 nm. At each time point, all samples were normalized to the placebo MP group without TGFβ1. Cell migration was visualized by fluorescent nuclear staining using 4′, 6-diamidino-2-phenylindole, dihydrocholoride (DAPI) (Sigma-Aldrich, St. Louis, Mo.) and observed in PBS under fluorescence (Leica DMIRB, Leica Microsystems, Bannockburn, Ill.) with appropriate filters.

Commercially pure Ti was cast into a hollow implant cylinder (FIG. 2a) with a dimension of 7×6 mm (dia.×1.) for in vitro studies. TGFβ1 encapsulated MPs or placebo MPs were infused in a gelatin sponge (Gelfoam) by negative pressure (FIG. 2a), which, in turn, was placed in the hollow core of the Ti implant (FIG. 2a). The hollow Ti implant with microencapsulated TGFβ1 or placebo MPs was placed in hMSC culture (FIG. 2a). Microparticles were observed inside the hollow Ti implant up to the tested 28 days (FIG. 2b). Adjacent to the outer wall of the hollow Ti implant, abundant hMSC accumulated in response to control-released 1 ng/mL TGFβ1 at 28 days (FIG. 2c). DAPI nuclear staining visualized the number of hMSC that had migrated into the gelatin sponge from the underlying cell culture against gravity, indicating the chemotactic effects of control-released TGFβ1. By day 28, there were abundant hMSC in the gelatin sponges infused with microencapsulated TGFβ1 at either 0.1 ng/mL (FIG. 2e) or 1 ng/mL (FIG. 2f), although cell migration also occurred in the TGFβ1-free sample (FIG. 2d). The metabolic activity of hMSC that had migrated into the gelatin sponges was significantly higher at 14 days for control-released 0.1 ng/mL TGFβ1 (n=6, p<0.05), and at 28 days for either 0.1 ng/mL or 1 ng/mL TGFβ1 (n=6, p<0.05) than TGFβ1-free group (FIG. 2g), suggesting that control-released TGFβ1 upregulates the metabolic activity of the migrated hMSC. Despite the apparently spontaneous migration of hMSC into the gelatin sponge without TGFβ1, the chemotaxized hMSC into the gelatin sponge by control-released TGFβ1 are metabolically more active.

### Example 4

### In Vivo Implantation Of TGFβ1-Encapsulated Microparticles in Porous Ti Implant

### In Vivo Implantation of Porous Titanium Implants

All animal procedures were approved by the local TACUC. Custom fabricated Ti implants were made with a dimension of 4×2.8 mm (1×dia.), 0.8 mm pores and a hollow inner core (1.8 mm dia.). A gelatin sponge was inserted into the hollow core of Ti implant and contained the following TGFβ1 doses: 5 mg high-density TGFβ1 MPs 100 ng) infused by negative pressure, adsorption of 100 ng or 1 μg TGFβ1 in gelatin sponges by overnight soaking (Linkhart, T. A., Mohan, S. & Baylink, D. J. Growth factors for bone growth and repair: IGF, TGF beta and BMP. Bone 19, 1S-12S (1996)), or 5 mg placebo MPs encapsulating PBS. The porous Ti implants were surgically implanted into the humeri of skeletally mature New Zealand white rabbits (3.5-4.0 kg) using aseptic technique under general anesthesia, similar to a previous approach (Sumner, D. R., Turner, T. M., Urban, R. M., Virdi, A. S. & Inoue, N. Additive enhancement of implant fixation following combined treatment with rhTGF-beta2 and rhBMP-2 in a canine model. J. Bone Joint Surg. Am. 88, 806-817 (2006)). The rabbit proximal humerus was chosen instead of more traditional models such as the tibia or femur (Alhadlaq, A. et al. Adult stem cell driven genesis of human-shaped articular condyle. Ann. Biomed. Eng. 32, 911-923 (2004); Schmidmaier, G. et al. Local application of growth factors (insulin-like growth factor-1 and transforming growth factor-betal) from a biodegradable poly(D,L-lactide) coating of osteosynthetic implants accelerates fracture healing in rats. Bone 28, 341-350 (2001)), because of low incidence of bone fracture of the humerus. An incision of approximately 3 cm was made in the shoulder region, with the subcutaneous soft tissue deflected, and the periosteum stripped using a periosteal elevator. Using a rotary handpiece (Straumann, Andover, Mass.) at no more than 1000 rpm, pilot holes of increasing diameter (2.2-2.8 mm) were drilled unicortically into the medullary cavity. Ti implants were press-fit, followed by wound closure. The same procedure was then repeated on the contralateral side for the placement of identical implants, given that growth factors delivered in one limb may effect on the contralateral limb (Hunziker, E. B. & Rosenberg, L. C. Repair of partial-thickness defects in articular cartilage: cell recruitment from the synovial membrane. J Bone Joint Surg. Am. 78, 721-733 (1996)). Animals were allowed normal cage activity during the entire healing process. Calcein blue (30 mg/kg) was injected subcutaneously at 3 wks to label newly formed bone 39 (Yamamoto, M. et al. Bone regeneration by transforming growth factor betal released from a biodegradable hydrogel. J Control Release 64, 133-142 (2000)).

Commercially pure Ti was cast into porous cylinder implants with a dimension of 2.8×4 mm (dia.×1.), a hollow inner core (1.8 mm dia.) and pores on the Ti wall (0.8 mm dia.) for in vivo implantation (FIG. 3a). TGFβ1-encapsulated or placebo MPs were infused in a gelatin sponge (Gelfoam) by negative pressure (FIG. 3b), which, in turn, was placed in the hollow core of the porous Ti implant. The porous Ti implants were implanted unicortically in the humerus bones of skeletally mature New Zealand white rabbits aseptically under general anesthesia (FIG. 3c). Following 4 wk in vivo implantation, Ti implants were found firmly integrated with host bone by radiographic examination (FIG. 3d) and remained integrated after embedding in methymetacrylate and bi-section of the Ti implant with a diamond knife (FIG. 3e). Interconnecting pores of the Ti implant are visible (FIG. 3e).

### Tissue Harvesting, Preparation and Analysis

At 4 wks post surgery, implant samples were removed with surrounding bone en bloc and embedded in methyl methacrylate. Samples were trimmed using a diamond saw and polished using diamond paper to 5 μm on a grinder/polisher system (Trizact, 3M, St. Paul, Minn.). For secondary ion scanning electron microscopy, samples were sputter coated with platinum/palladium (Pt/Pd) metal films of approximately 3 nm and imaged under high voltage and constant pressure (Hitachi S-3000N Variable Pressure-SEM, Tokyo, Japan). BIC and bone volume to tissue volume (BV/TV) within 0.8 mm pores of the porous Ti implants were quantified using computerized image analysis software (Yamamoto, M. et al. Bone regeneration by transforming growth factor betal released from a biodegradable hydrogel. J Control Release 64, 133-142 (2000)), (ImagePro Plus, Media Cybernetics, Silver Spring, Md.). Microcomputed tomography (μCT) (Scanco 40, Wayne, Pa.) was used to scan bone-implant samples at intervals that correspond to a resolution of ≈20 μm in plane and slice thickness of 20 μm (Moioli, E. K., Hong, L. & Mao, J. J. Inhibition of osteogenic differentiation of human mesenchymal stem cells. Wound Repair Regen. 15, 413-424 (2007)) For histology, samples were glued to plastic slides, ground to thin sections using diamond paper, and stained using hematoxylin and eosin (H&E), per a prior approach (Yamamoto, M. et al. Bone regeneration by transforming growth factor betal released from a biodegradable hydrogel. J Control Release 64, 133-142 (2000)).

### Example 5

### Controlled-Release TGFβ1 From Porous Ti Implant Significantly Augments Bone-to-Implant Contact and Bone Ingrowth in Vivo

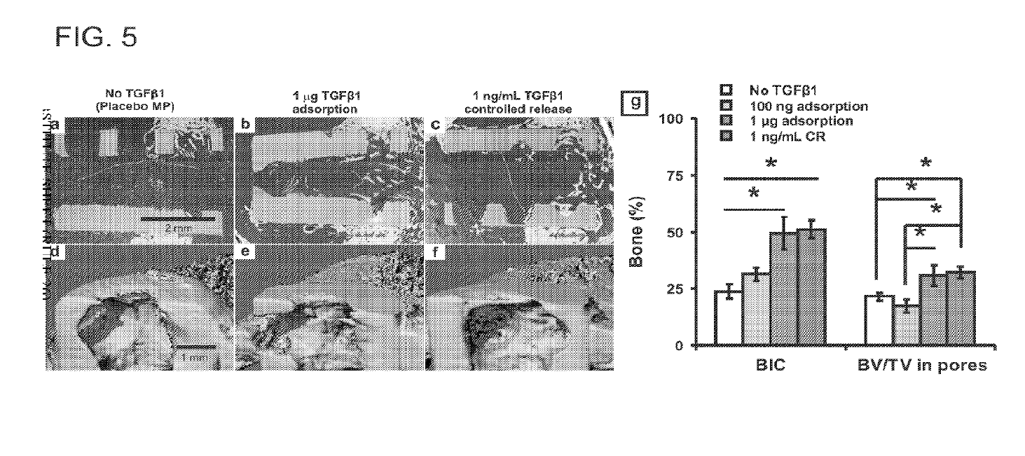

In comparison to moderate bone-to-implant contact in the TGFβ1-free implant (placebo MPs) (FIG. 3f) or 100 ng/mL gelatin-adsorbed TGFβ1 implant (FIG. 3g), there was substantial BIC for both 1 μg gelatin-adsorbed TGFβ1 implant (FIGS. 3h) and 1 ng/mL control-released TGFβ1 implant (FIG. 3i). The total amount of control-released 1 ng/mL TGFβ1 for the tested 4 wks of in vivo implantation is calculated to be 100 ng, since 19.11±3.50 ng microencapsulated TGFβ1/mg TGFβ1 MPs×5 mg implanted TGFβ1 MPs 100 ng TGFβ1. Thus, 1 ng/mL control-released TGFβ1 is as effective as 1 μg gelatin-adsorbed TGFβ1, but at a 10-fold lower drug dose. Similarly, both 1 μg gelatin-adsorbed TGFβ1 (FIGS. 3l) and 1 ng/mL microencapsulated TGFβ1 (FIG. 3m) induced marked bone ingrowth in the pores of Ti implants, in comparison to moderate bone ingrowth without TGFβ1 (FIG. 3j) or with 100 ng gelatin-adsorbed TGFβ1 (FIG. 3k). These qualitative findings are substantiated below by SEM (FIG. 5a-c) and μCT imaging (FIG. 5d-f) of bone ingrowth, and further by quantitative, computerized histomorphometry of BIC and bone ingrowth (FIG. 5g).

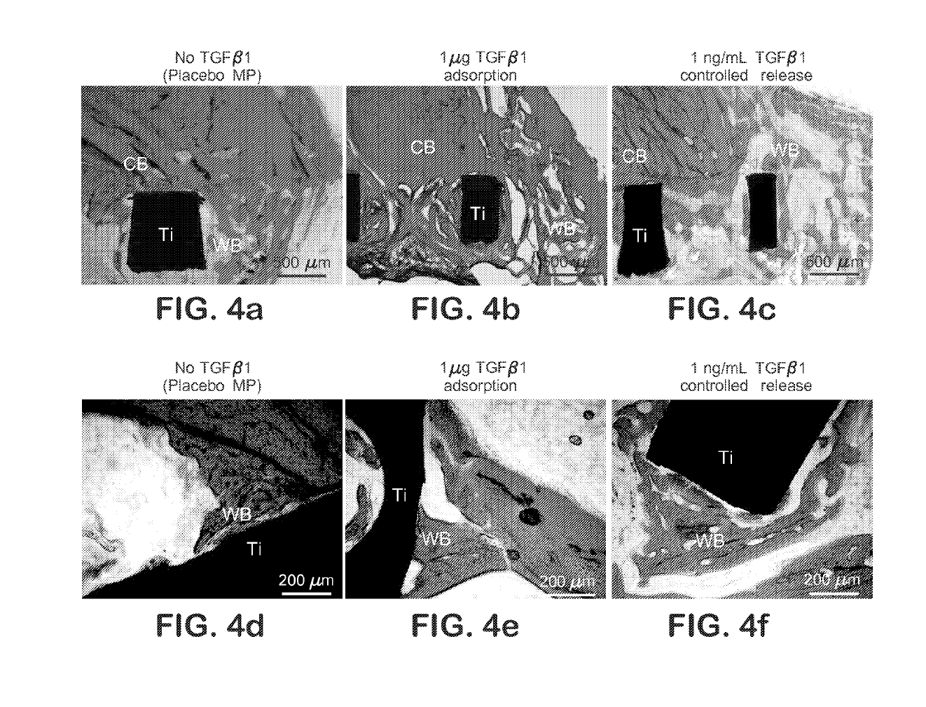

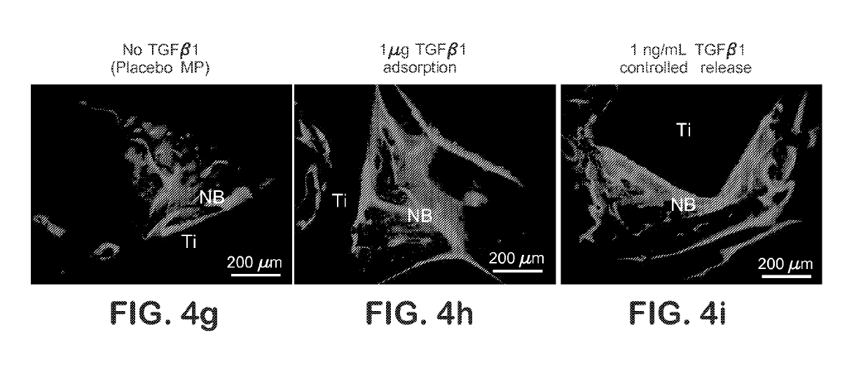

Further examination reveals the ingrowth of substantial woven bone (WB) that was integrated with cortical bone (CB) for both 1 μg gelatin-adsorbed TGFβ1 implant (FIG. 4b) and 1 ng/mL control-released TGFβ1 implant (FIG. 4c), in comparison to moderate WB formation in the TGFβ1-free implant (FIG. 4a). The newly formed WB was surrounded by bone marrow cavities (FIGS. 4d-f), known as a source of osteoprogenitor cells and/or mesenchymal stem cells (Holland, T. A. et al. Degradable hydrogel scaffolds for in vivo delivery of single and dual growth factors in cartilage repair. Osteoarthritis Cartilage 15, 187-197 (2007); Moioli, E. K., Hong, L., Guardado, J., Clark, P. A. & Mao, J. J. Sustained release of TGFbeta3 from PLGA microspheres and its effect on early osteogenic differentiation of human mesenchymal stem cells. Tissue Eng. 12, 537-546 (2006)). Calcein labeling revealed marked new bone formation for both 1 μg gelatin-adsorbed TGFβ1 implant (FIG. 4h) and 1 ng/mL microencapsulated TGFβ1 implant (FIG. 4i), in comparison to moderate new bone formation adjacent to the TGFβ1-free Ti implant (FIG. 4g).

Scanning electron microscopy (SEM) showed marked BIC and WB formation in the surface and pores of both 1 μg gelatin-adsorbed TGFβ1 implant (FIGS. 5b) and 1 ng/mL control-released TGFβ1 implant (FIG. 5c), in comparison with the TGFβ1-free or placebo MP implant (FIG. 5a). Micro-computed tomography (μCT) revealed marked bone ingrowth into the pores of Ti implants for both 1 μg gelatin-adsorbed TGFβ1 (FIGS. 5e) and 1 ng/mL microencapsulated TGFβ1 (FIG. 5f), in comparison with the TGFβ1-free or placebo MP implant (FIG. 5d). BIC and BV/TV within the implant's pores were quantified using computerized histomorphometry, per previous methods (Moioli, E. K., Hong, L. & Mao, J. J. Inhibition of osteogenic differentiation of human mesenchymal stem cells. Wound Repair Regen. 15, 413-424 (2007); Alhadlaq, A. et al. Adult stem cell driven genesis of human-shaped articular condyle. Ann. Biomed. Eng. 32, 911-923 (2004)). Both BIC and BV/TV for control-released 1 ng/mL TGFβ1 were significantly higher (46±16% and 29±9.6%, respectively) than placebo MPs (24±8% and 19±6.2%, respectively) (p<0.05, n=6) (FIG. 5g), representing 96% increase in BIC and 50% increase in BV/TV. Importantly, the BIC and BV/TV yielded by 1 ng/mL control-released TGFβ1 showed no significant differences from 1 μg gelatin-adsorbed TGFβ1 (BIC: 49±19%, BV/TV: 31±11%) (p<0.01, n=6) (FIG. 5g), again suggesting that controlled release is effective at a 10-fold less drug dose than adsorption.

1-16. (canceled)

17. A porous, implantable medical device comprising:
a device body;
a plurality of pores contacting a surface of the device body;
at least one bioactive cue; and
a biocompatible controlled release encapsulation material;
wherein
the at least one bioactive cue is encapsulated in the encapsulation material; and
the at least one encapsulated bioactive cue is contained (i) within at least one of the plurality of pores or (ii) within the device body in connection with at least one of the plurality of pores.

18. The device of claim 17, wherein at least a portion of the plurality of pores are interconnected, the interconnected pores forming throughbores connecting an inner surface of the device body and an outer surface of the device body.

19. The device of claim 17, wherein
the encapsulated bioactive cue is not biologically-available until release; and
the encapsulated bioactive cue has a temporal or spatial release profile.

20. The device of claim 17, wherein at least a portion of the device body is hollow.

21. The device of claim 20, further comprising
a biocompatible matrix;
optionally, a tissue progenitor cell; and
optionally, an immunomodulative agent;
wherein
the biocompatible matrix is contained in the hollow portion of the device body; and
the tissue progenitor cell, where present, or the immunomodulative agent, where present, is contained in or on the biocompatible matrix.

22. The device of claim 17, wherein the pores are of a non-uniform size.

23. The device of claim 17, wherein the at least one bioactive cue is encapsulated in biocompatible controlled release encapsulation material selected from the group consisting of: polylactic acid (PLA); polyglycolid acid (PGA); copolymers of lactic acid and glycolic acid (PLGA); polycaprolactone; polyphosphoester; polyorthoester; poly(hydroxy butyrate); poly(diaxanone); poly(hydroxy valerate); poly(hydroxy butyrate-co-valerate); poly(glycolide-co-trimethylene carbonate); polyanhydrides; polyphosphoester; poly(ester-amide); polyphosphoeser; polyphosphazene; poly(phosphoester-urethane); poly(amino acids); polycyanoacrylates; biopolymeric molecules such as fibrin; fibrinogen; cellulose; starch; collagen; and hyaluronic acid; or a mixture or a copolymer thereof.

24. The device of claim 17, wherein the at least one bioactive cue is selected from the group consisting of: a growth factor; a cytokine; DNA; RNA; a transcription factor; a tissue ingrowth modulator; a tissue adhesion modulator; a chemotherapeutic agent; an immunomodulative agent; and a tissue progenitor cell.

25. The device of claim 17, wherein the at least one bioactive cue is selected from the group consisting of: activin A, adrenomedull in, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, an cGMP analog, ChDI, CLAF, claudins, collagen, collagen receptor α₁β₁, collagen receptor α₂β₁, connexins, Cox-2, ECDGF, ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1, ephrins, Epo, HGF, TNF-alpha, TGF-beta, PD-ECGF, PDGF, IGF, IL8, growth hormone, fibrin fragment E, FGF-5, fibronectin, fibronectin receptor α₅β₁, Factor X, HB-EGF, HBNF, HGF, HUAF, heart derived inhibitor of vascular cell proliferation, IFN-gamma, Il1, IGF-2 integrin receptor, integrin β subunit, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin, neurothelin, nitric oxide donors, nitric oxide synthases, notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPARγ ligand, phosphodiesterase, prolactin, prostacyclin, protein S, smooth muscle cell-derived growth factor, smooth muscle cell-derived migration factor, sphingosine-1-phosphate-1, Syk, SLP76, tachykinins, TGF-β, Tie 1, Tie2, TGF-β receptor, TIMPs, TNF-alpha, TNF-beta, transferrin, thrombospondin, urokinase, VEGF-A, VEGF-B, VEGF-C, VEGF-D, VEGF-E, VEGF, VEGF₁₆₄, VEGI, EG-VEGF, VEGF receptors, PF4, 16 kDa fragment of prolactin, prostaglandins E1 and E2, steroids, heparin, 1-butyryl glycerol, and nicotinic amide

26. The device of claim 17, wherein the at least one bioactive cue is TGFβ1; BMP2; or TGFβ1 and BMP2.

27. The device of claim 17, comprising a plurality of bioactive cues.

28. The device of claim 17, comprising:
a biocompatible matrix;
wherein
at least a portion of the plurality of pores are interconnected, the interconnected pores forming throughbores connecting an inner surface of the device body and an outer surface of the device body;
at least a portion of the device body is hollow;
the biocompatible matrix is contained in the hollow portion of the device body;
the at least one bioactive cue comprises TGFβ1; BMP2; or TGFβ1 and BMP2;
the at least one bioactive cue is contained in or on the biocompatible matrix;
the encapsulated bioactive cue is not biologically-available until release;
the encapsulated bioactive cue has a temporal or spatial release profile; and
a released bioactive cue has a diffusion pathway from the hollow portion of the device body, through a throughbore of an interconnected pore to an outer surface of the device body.

29. A method of preparing a porous, implantable medical device comprising:
a) providing a porous, implantable medical device, the device comprising (i) a device body and (ii) a plurality of pores contacting a surface of the device body;
b) encapsulating at least one bioactive cue in a biocompatible controlled release encapsulation material; and
c) introducing the at least one encapsulated bioactive cue into (i) at least one pore of the plurality of pores or (ii) into the device body in connection with at least one of the plurality of pores.

30. The method of claim 29, wherein at least a portion of the plurality of pores are interconnected, the interconnected pores forming throughbores connecting an inner surface of the device and an outer surface of the device.

31. The method of claim 19, wherein the at least one bioactive cue is not biologically-available until release; and the biocompatible controlled release encapsulation material comprises a time-controlled or spatial-based release encapsulation material.

32. The method of claim 29, wherein at least a portion of the device body is hollow.

33. The method of claim 29, wherein the pores are of a non-uniform size.

34. The method of claim 29, wherein the at least one bioactive cue is encapsulated in biocompatible controlled release encapsulation material selected from the group consisting of: polylactic acid (PLA); polyglycolid acid (PGA); copolymers of lactic acid and glycolic acid (PLGA); polycaprolactone; polyphosphoester; polyorthoester; poly(hydroxy butyrate); poly(diaxanone); poly(hydroxy valerate); poly(hydroxy butyrate-co-valerate); poly(glycolide-co-trimethylene carbonate); polyanhydrides; polyphosphoester; poly(ester-amide); polyphosphoeser; polyphosphazene; poly(phosphoester-urethane); poly(amino acids); polycyanoacrylates; biopolymeric molecules such as fibrin; fibrinogen; cellulose; starch; collagen; and hyaluronic acid; or a mixture or a copolymer thereof.

35. The method of claim 29, wherein the at least one bioactive cue is selected from the group consisting of: a growth factor; a cytokine; DNA; RNA; a transcription factor; a tissue ingrowth modulator; a tissue adhesion modulator; a chemotherapeutic agent; an immunomodulative agent; and a tissue progenitor cell.

36. A method for treating a subject having a tissue or organ defect, comprising:
implanting the porous, implantable medical device of claim 17 into a subject in need thereof;
optionally, determining an appropriate bioactive cue according to a diagnosis of the subject;
optionally, providing the appropriate bioactive cue encapsulated in a biocompatible controlled release encapsulation material; and
optionally, introducing the encapsulated bioactive cue into a pore of the implantable medical device.
